(19) United States
(12) Patent Application Publication
(10) Pub. No.: US 2020/0281701 A1
(43) Pub. Date: Sep. 10, 2020

(54) THREE-DIMENSIONAL INTRAORAL SCANNER

(71) Applicant: Tae Hoon Kim, Santa Monica, CA (US)

(72) Inventor: Tae Hoon Kim, Santa Monica, CA (US)

(21) Appl. No.: 16/810,635

(22) Filed: Mar. 5, 2020

Related U.S. Application Data
(60) Provisional application No. 62/814,588, filed on Mar. 6, 2019.

(51) Int. Cl. A61C 9/00; A61B 5/00; G06T 7/00

(52) U.S. Cl. CPC A61C 9/0053; A61B 5/682; G06T 7/0012; G06T 2200/04; G06T 2207/30036; G06T 2207/10132; A61B 2562/0233

(57) ABSTRACT

A three-dimensional intraoral scanner includes: an outer body forming a trough, an outer surface of the outer body comprising a plastic material and an inner surface of the outer body at the trough including a transparent or translucent material, the trough being configured to fit around a row of a patient's teeth; and a sled within the outer body and configured to move along the trough, the sled including a plurality of ultrasonic sensors and a plurality of optical sensors configured to image the patient's mouth.

### CROSS-REFERENCE TO RELATED APPLICATIONS

This application claims priority to and the benefit of U.S. Provisional Application Ser. No. 62/814,588, filed on Mar. 6, 2019, the entire content of which is incorporated herein by reference.

### BACKGROUND

### 1. Field

Aspects of example embodiments of the present disclosure relate to a three-dimensional intraoral scanner.

### 2. Related Art

Previously, traditional x-ray machines have been used to take images of a patient's tooth and tooth root structure and condition. The method, however, has drawbacks, including a cumbersome, uncomfortable, and labor-intensive process to take x-rays, many of which are necessary to image all parts of a patient's oral cavity (or mouth). Further, the resulting images are merely two-dimensional representations of the patient's mouth and the only available angle from which to view the patient's teeth is from the outside of the patient's mouth toward the inside.

To overcome some of these deficiencies, intraoral scanners have been developed. These related-art intraoral scanners, however, must be manually moved around a patient's mouth to get a sufficient number of images or data points to create a three-dimensional representation of the patient's mouth. This process is cumbersome, labor-intensive, and may take from about 10 to about 15 minutes to complete. Further, these related-art intraoral scanners may only include optical sensors configured to sense visible light, meaning the resulting three-dimensional representation only shows the patient's visible teeth and gum condition, not the underlying root condition.

### SUMMARY

The present disclosure is directed toward various embodiments of a three-dimensional intraoral scanner that uses both optical and ultrasonic images to rapidly provide a three-dimensional representation of a patient's oral cavity, including the visible teeth and gum condition along with the underlying root condition.

This summary is provided to introduce a selection of features and concepts of example embodiments of the present disclosure that are further described below in the detailed description. This summary is not intended to identify key or essential features of the claimed subject matter nor is it intended to be used in limiting the scope of the claimed subject matter. One or more of the described features according to one or more example embodiments may be combined with one or more other described features according to one or more example embodiments to provide a workable method or device.

### BRIEF DESCRIPTION OF THE DRAWINGS

FIG. 1 is a representation of a patient's mouth with a cutaway showing a tooth's underlying root structure;

FIG. 2 is a perspective view of a three-dimensional intraoral scanner according to an embodiment of the present disclosure;

FIGS. 3 and 4 show a process of using the three-dimensional intraoral scanner shown in FIG. 2 according to an embodiment of the present disclosure;

FIG. 5 is a perspective view of a three-dimensional intraoral scanner according to another embodiment of the present disclosure;

FIGS. 6-8 show aspects of a three-dimensional intraoral scanner according to another embodiment of the present disclosure; and

FIG. 9 shows a three-dimensional intraoral scanner according to another embodiment of the present disclosure;

FIG. 10 shows a three-dimensional intraoral scanner according to another embodiment being moved along a patient's mouth;

FIGS. 11 and 12 show interchangeability between a wireless module and a corded connection;

FIGS. 13-15 show covers for the three-dimensional intraoral scanners; and

FIGS. 16 and 17 show a three-dimensional intraoral scanner according to another embodiment of the present disclosure; and

FIG. 18 shows examples of a point cloud of a patient's mouth, a dense point cloud of the patient's mouth, a three-dimensional representation of the patient's mouth, and a three-dimensional representation of the patient's mouth with texturing and shading.

### DETAILED DESCRIPTION

The detailed description set forth below in connection with the appended drawings is intended as a description of example embodiments of the present disclosure and is not intended to represent the only forms in which the present disclosure may be embodied. The description sets forth aspects and features of the present disclosure in connection with the illustrated embodiments. It is to be understood, however, that the same or equivalent aspects and features may be accomplished by different embodiments, and such other embodiments are encompassed within the spirit and scope of the present disclosure. As noted elsewhere herein, like element numbers in the description and the drawings are intended to indicate like elements. Further, descriptions of features, configurations, and/or other aspects within each embodiment should typically be considered as available for other similar features, configurations, and/or aspects in other embodiments.

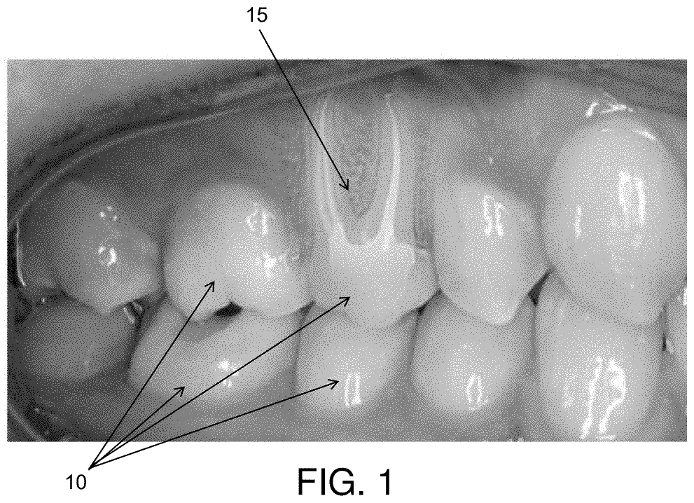

Referring to FIG. 1, a mouth is shown with a plurality of teeth 10. One of the teeth 10 has a “cut-away” view showing the underlying root structure 15 of the tooth 10, such as what could be viewed by using, for example, an x-ray. To accurately diagnose a patient, the condition of both the tooth 10 and the underlying root structure 15 should be examined. Traditionally, diagnosing a patient included both a direct physical examination of the patient's mouth to inspect the visible teeth and gums along with a number of x-ray images to inspect the inner portions of the tooth and the underlying root structures. This multi-step process is tedious, costly, and labor-intensive.

Recently, intraoral scanners have been developed. However, these related-art intraoral scanners must be moved (or swept) along a patient's mouth to take a sufficient number of images to produce a three-dimensional representation (or model) of a patient's mouth and may only include optical sensors such that only the visible aspects of a patient's teeth and gums can be represented. A technician must be trained to use these related-art scanners, as they generally cannot be moved too quickly or the resulting images will be distorted and three-dimensional representations (or models) will be inaccurate with flaws and missing information, and they must be maintained at a proper orientation to ensure all relevant aspects of a patient's mouth are captured. Further, x-rays may still be used to diagnose the condition of underlying roots, which is otherwise not visible when using only optical sensors. Although these related-art intraoral scanners may be less cumbersome than the previous physical examination and x-rays, they are still labor intensive and time consuming and still require a multi-step process for diagnosing a patient.

According to embodiments of the present disclosure, a three-dimensional intraoral scanner is provided that concurrently (or simultaneously) takes both optical images and ultrasonic images of all relevant aspects of a patient's oral cavity (or mouth) within about one to two seconds. For example, the three-dimensional intraoral scanner may have a mouth guard or mouth tray form (or shape) that sits between a patient's upper and lower teeth and extends along all of (or substantially all of) the patient's teeth. Because the three-dimensional intraoral scanner is embodied as a mouth guard or mouth tray, there is no need for a technician to move (or sweep) the three-dimensional intraoral scanner along a patient's mouth, thereby allowing an untrained or lesser trained technician to use the three-dimensional intraoral scanner and allowing the scanning to occur much faster. Further, because the three-dimensional intraoral scanner includes both optical and ultrasonic sensors, the resulting three-dimensional representation may overlay both the visible tooth and gum condition with the underlying root condition.

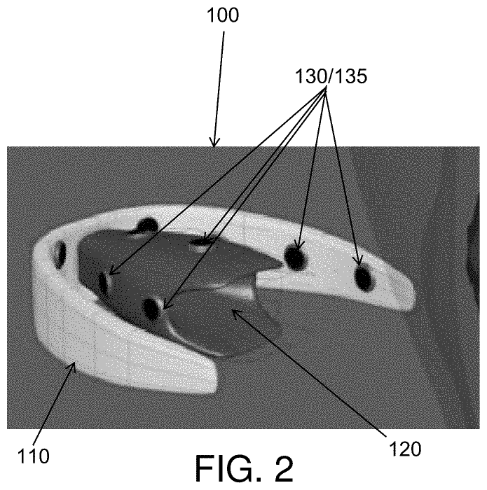

Referring to FIG. 2, a three-dimensional intraoral scanner 100 is shown. The three-dimensional intraoral scanner 100 may include an outer U-shaped portion 110 and an inner portion 120 connected to the outer U-shaped portion 110. In use in a patient's mouth, the outer U-shaped portion 110 may sit outside of (e.g., may sit along an outer surface of) the patient's teeth and gums and the inner portion 120 may sit inside of (e.g., may sit along an inner surface of) the patient's teeth and gums. The patient may hold the three-dimensional intraoral scanner 100 in his or her mouth by clamping down on the inner portion 120 of the three-dimensional intraoral scanner 100.

In different embodiments, the size of the three-dimensional intraoral scanner 100 may be suitably varied for use with adult patients and child patients. That is, the three-dimensional intraoral scanner 100 may be formed in different sizes to fit adult patients and child patients.

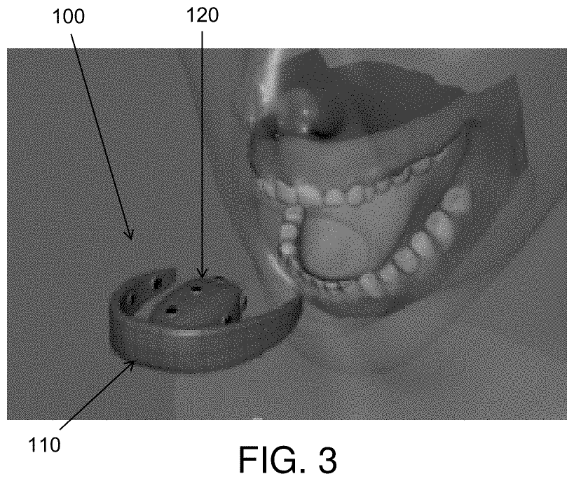

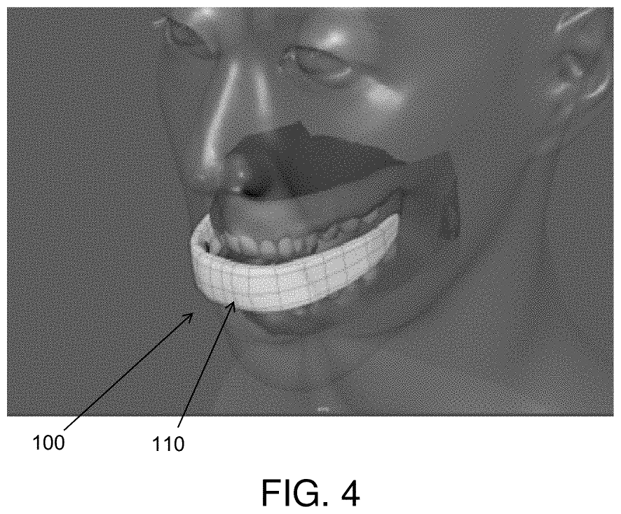

Referring to FIGS. 2-4, the three-dimensional intraoral scanner 100 includes a plurality of optical and ultrasonic sensors (e.g., optical and ultrasonic cameras) 130/135. The sensors 130/135 may be arranged on an inner surface of the outer U-shaped portion 110 and on an outer surface of the inner portion 120. In some embodiments, additional sensors 130/135 may be formed on upper and lower surfaces of the inner portion 120 to capture images of the hard palate (e.g., the roof), the floor of a patient's mouth, and the patient's tongue.

The placement of the sensors 130/135 in FIGS. 2-4 is schematically illustrated for ease of description and understanding. The sensors 130/135 may be suitably arranged on the three-dimensional intraoral scanner 100 to capture images of the entire oral cavity (or mouth). Accordingly, the position of the sensors 130/135 is merely shown for reference, and the positions of the sensors 130/135 may be suitably adjusted.

In some embodiments, the optical cameras 130 may be structured light cameras, Time-of-Flight (ToF) depth sensors, and/or other depth sensors, and may use polarization to prevent or substantially reduce instances of specular reflection. For example, the optical cameras 130 may have polarization filters (e.g., polarizers) arranged over the lens to prevent or substantially reduce specular reflections, which may occur when imaging teeth because they are often wet with saliva.

The ultrasonic sensors 135 may be configured to image a patient's tooth root structure. Because tooth roots are generally covered by gum tissue, except when a patient is suffering periodontitis (gum disease), the optical sensors 130 are generally unable to image the teeth root structures. The ultrasonic sensors 135, however, are able to penetrate gum tissue by using ultrasonic waves, which reflect from the relatively hard or dense root structures but pass through the relatively soft gum tissue. Thus, by using the combination of optical and ultrasonic sensors 130/135, a composite three-dimensional representation of a patient's visible tooth structure and non-visible tooth root structure may be generated.

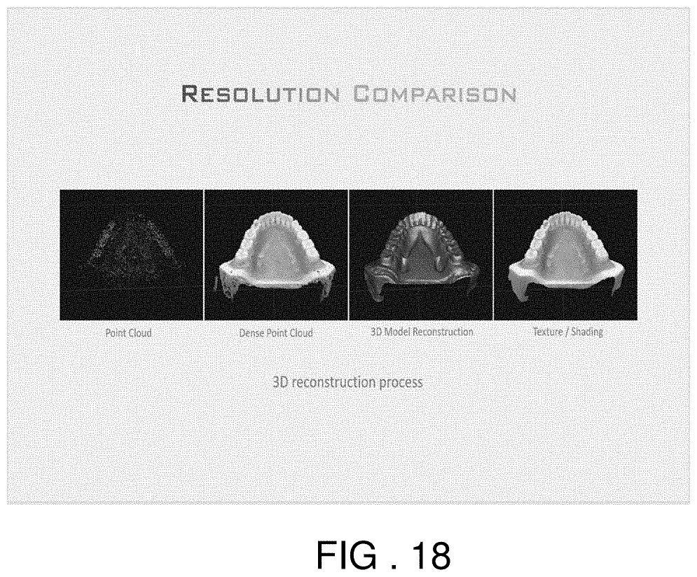

In some embodiments, a computer vision system (or computer vision algorithm) may use the received optical and ultrasonic images to create a three-dimensional representation (or model) of a patient's mouth. For example, the computer vision system may utilize a machine learning algorithm to colorize the received ultrasonic images for consistency with the received optical images, to accurately orient the received optical information with the received ultrasonic information, to create a point cloud (e.g., a set of data points in a three-dimensional space), and/or to create triangulated three-dimensional mesh with color textures. FIG. 18 shows examples of a point cloud of a patient's mouth, a dense point cloud of the patient's mouth, a three-dimensional representation (or model) of the patient's mouth, and a three-dimensional representation of the patient's mouth with texturing and shading.

For example, rather than calibrating each three-dimensional intraoral scanner 100 to compensate for differences in manufacturing (e.g., manufacturing tolerances, optical and ultrasonic sensor alignment, etc.), the computer vision system may be used to interpret the received information and to generate an accurate three-dimensional representation of a patient's mouth.

In some embodiments, the three-dimensional intraoral scanner 100 may include a battery (e.g., a rechargeable battery) inside the outer U-shaped portion 110 and/or inside the inner portion 120, and the three-dimensional intraoral scanner 100 may include a wireless transceiver to wirelessly receive and transmit data to a nearby computer system. That is, for ease of use and patient comfort, the three-dimensional intraoral scanner 100, according to some embodiments, may be wireless.

The wireless transceiver may operate by using the Bluetooth® standard (Bluetooth is a registered trademark of BLUETOOTH SIG, INC., a Delaware Corporation). The battery may be charged by, for example, wireless charging, such as by induction charging or the like, such that a charging port may be omitted from the three-dimensional intraoral scanner 100. However, in some embodiments, a charging port, with or without a cover, may be included for charging the battery.

In some embodiments, a wire may be provided for powering the three-dimensional intraoral scanner 100 and for data transfer with a nearby computer system.

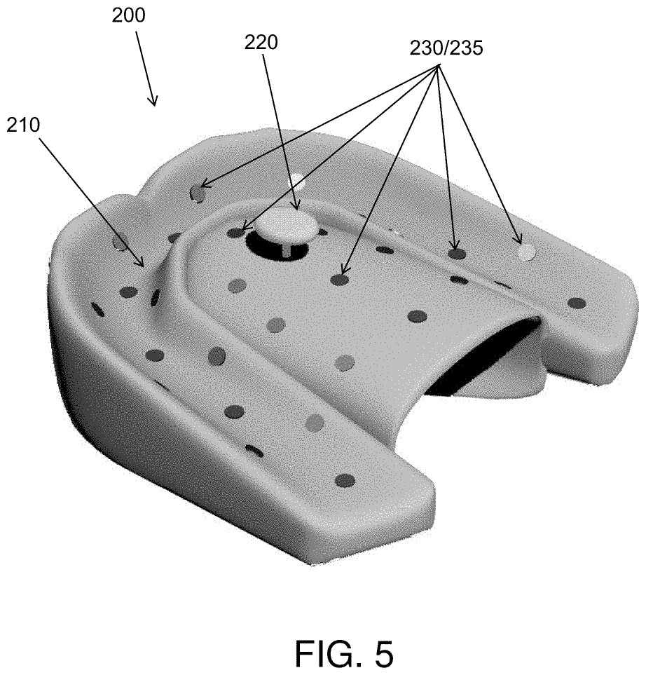

FIG. 5 is a perspective view of a three-dimensional intraoral scanner 200 according to another embodiment of the present disclosure. The three-dimensional intraoral scanner 200 may be a one-piece (e.g., integral) design that extends under and around a patient's teeth, different from the three-dimensional intraoral scanner 100 which was positioned on opposite sides of a patient's teeth but not under a patient's teeth.

A plurality of optical and ultrasonic sensors 230/235 may be arranged on the surface (e.g., on both surfaces) of the three-dimensional intraoral scanner 200. For example, some of the illustrated silver dots may be optical sensors 230 and other ones may be ultrasonic sensors 235, or in other embodiments, each of the silver dots may be both optical and ultrasonic sensors 230/235. The optical and ultrasonic sensors 230/235 are substantially similar to the optical and ultrasonic sensors 130/135 described above, and accordingly, a repeated description thereof will be omitted. Also, the position of the sensors 230/235 may be suitably varied from what is shown in FIG. 5 to capture images of a patient's oral cavity (or mouth).

In the three-dimensional intraoral scanner 200, sensors 230/235 may be arranged at a base of a trough 210 to capture images of tooth enamel. For example, because the three-dimensional intraoral scanner 100 shown in FIG. 2 does not extend under a patient's teeth, it may not be able to capture images of a patient's tooth enamel where cavities often form. The three-dimensional intraoral scanner 200, however, has a mouthpiece shape with the trough 210 that extends under the patient's teeth, thereby providing images (e.g., optical images) of the enamel of a patient's teeth to better assist with diagnosing cavities and the like.

The three-dimensional intraoral scanner 200 may also include a soft protrusion 220 that extends from an inner portion of the scanner 200. The protrusion 220 may contact a patient's hard palate (the roof of the mouth) to ensure proper spacing between the three-dimensional intraoral scanner 200 and the hard palate for better imaging and the like. In some embodiments, however, the protrusion 220 may be omitted.

Similar to the three-dimensional intraoral scanner 100, the three-dimensional intraoral scanner 200 may include a battery and a wireless transceiver for wireless operation. In other embodiments, however, a wired connection may be provided to power the three-dimensional intraoral scanner 200 and for data transfer between the three-dimensional intraoral scanner 200 and a nearby computer system.

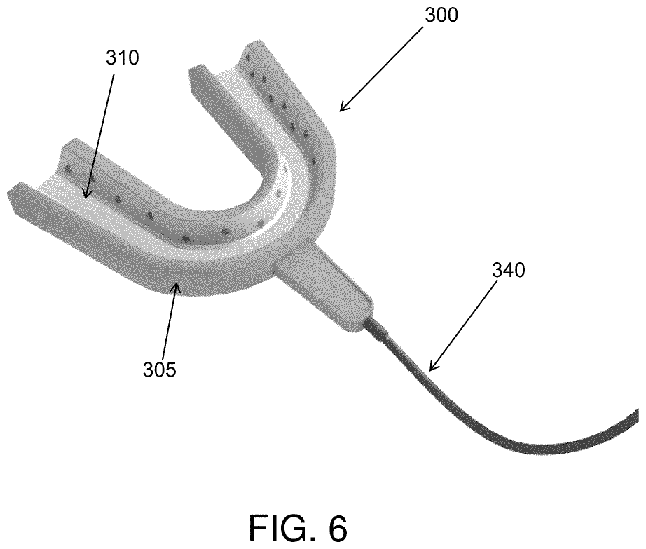

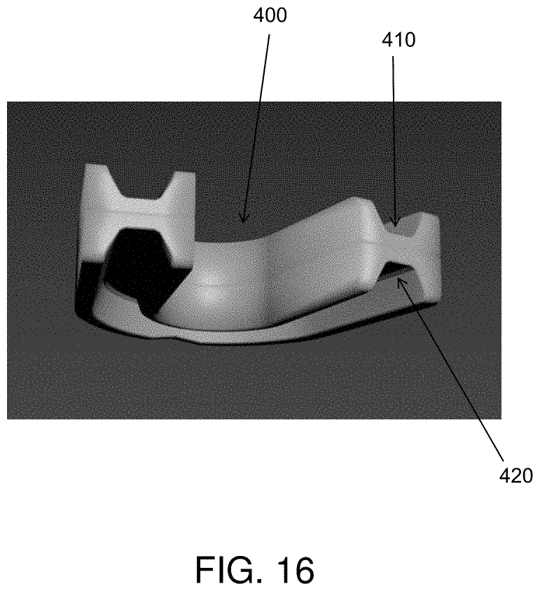

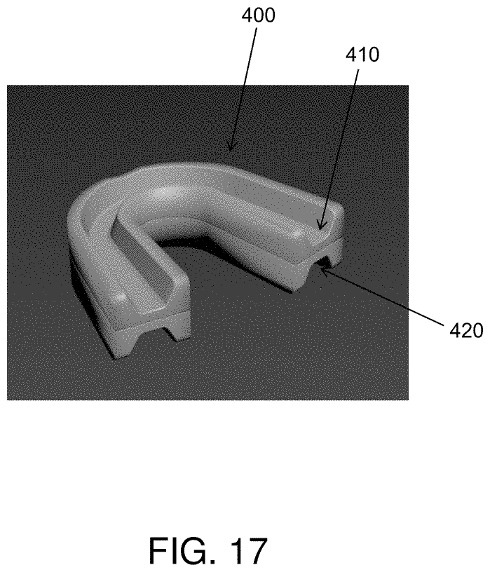

FIGS. 6-8 show aspects of a three-dimensional intraoral scanner 300 according to another embodiment of the present disclosure. The three-dimensional intraoral scanner 300 may have a single-trough shape 310 to fit around one row of a patient's teeth or a double-trough shape to fit around both upper and lower rows of a patient's teeth (see, e.g., FIGS. 16 and 17).

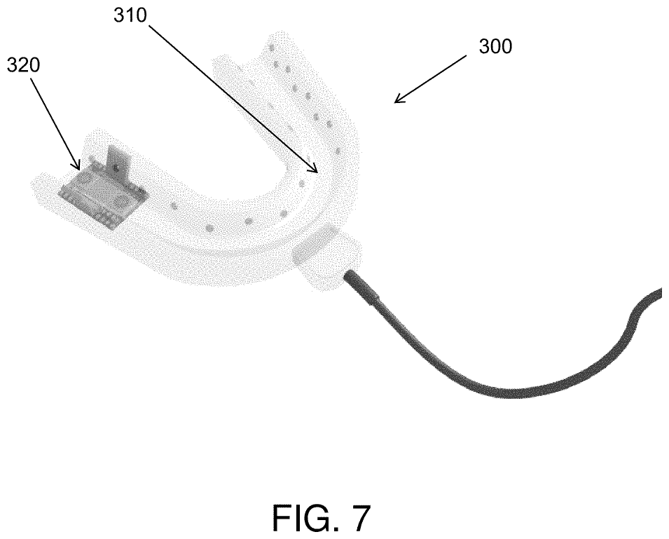

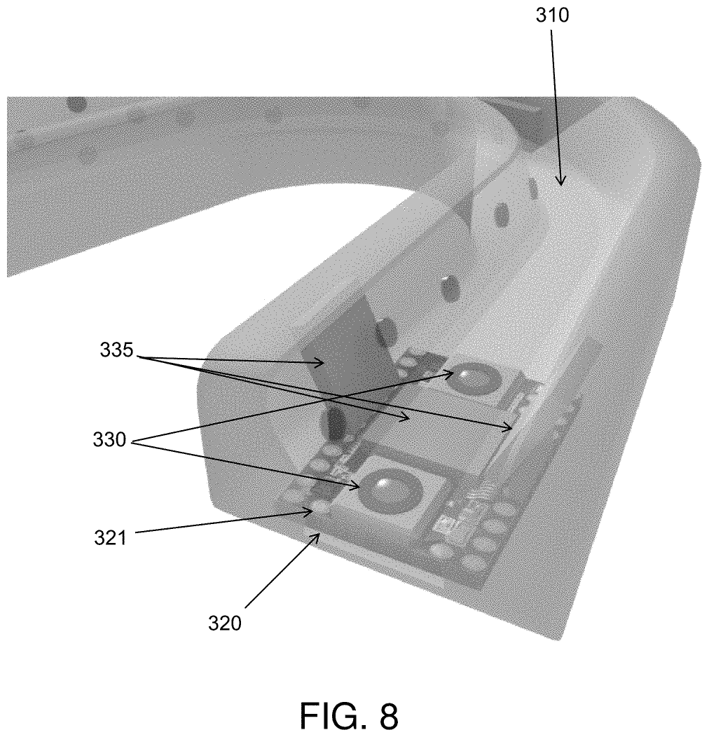

Referring to FIGS. 7 and 8, the three-dimensional intraoral scanner 300 may include a sled 320 that is configured to move along the trough 310. The sled 320 may include a circuit board 321, and a plurality of optical and ultrasonic sensors 330/335 may be arranged on the circuit board 321. By including the sled 320, the number of sensors 330/335 in the three-dimensional intraoral scanner 300 may be reduced when compared to the three-dimensional intraoral scanners 100/200 described above.

The sled 320 may move along the trough 310 by, for example, magnetic attraction and repulsion. In other embodiments, the sled 320 may move along the trough 310 by using a motor. The sled 320 may move along the entire length of the trough 310 in about one to about two seconds to image a patient's entire mouth.

In some embodiments, a plurality of sleds 320 may be provided in the three-dimensional intraoral scanner 300. For example, one sled 320 may be provided at one end of the trough 310 to move toward a patient's front teeth and another sled 320 may be provided at another end of the trough 310 to move toward the patient's front teeth. In such an embodiment, the scanning time may be reduced. For example, the scanning time may be half of the scanning time of an embodiment in which only one sled 320 is present.

In some other embodiments, when the three-dimensional intraoral scanner 300 has the double-trough shape, one or more sleds 320 may be provided in each of the upper trough to image a patient's upper teeth and corresponding root structure and the lower trough to image the patient's lower teeth and corresponding root structure.

The trough 310 may be made from (or may include) a glass or crystal material that is transparent or translucent to both visible light and ultrasonic waves. This way, as the sled 320 moves along the trough, the optical and ultrasonic sensors 330/335 may image the patient's teeth and underlying root structure while maintaining a barrier (e.g., a glass or crystal barrier) between the sled 320 and the patient's mouth. An outer body of the three-dimensional intraoral scanner 300 (e.g., the outer surfaces of the three-dimensional intraoral scanner 300) 305 may be, for example, a plastic or the like for increased durability and comfort.

The three-dimensional intraoral scanner 300 may include a cable 340 for powering the sensors 330/335 and for transmitting data to from the sensors 330/335 to a nearby computer system. However, in some embodiments, the three-dimensional intraoral scanner 300 may include a battery and a wireless transceiver.

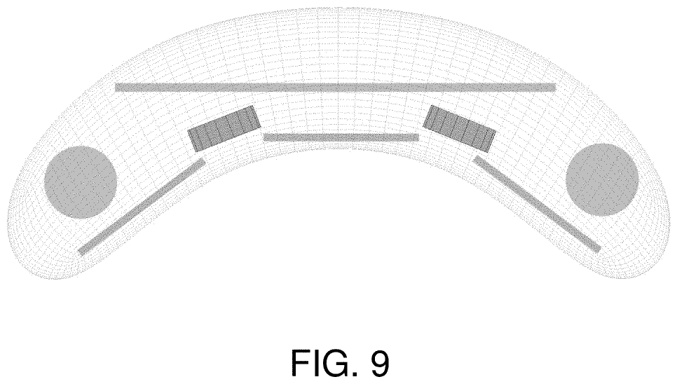

FIG. 9 shows a three-dimensional intraoral scanner according to another embodiment of the present disclosure. For example, the three-dimensional intraoral scanner shown in FIG. 9 may include a wireless transceiver for wireless connection to a computer vision system.

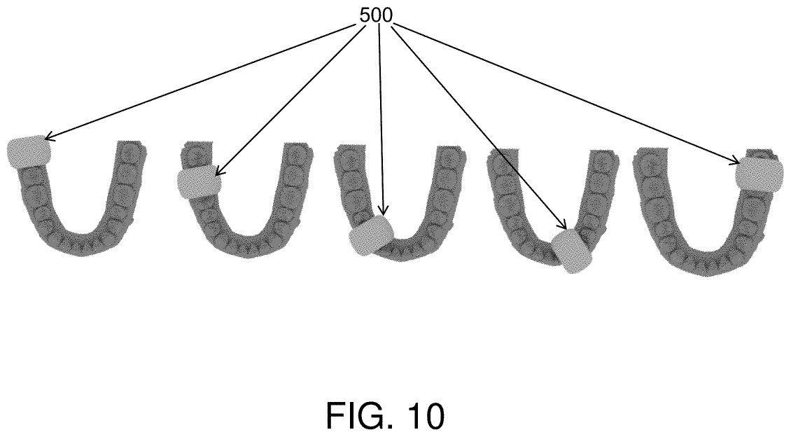

FIG. 10 shows a three-dimensional intraoral scanner 500 according to another embodiment being moved along a patient's mouth. The three-dimensional intraoral scanner 500 may be relatively small compared to the other embodiments described herein and may be used to scan only portions of a patient's mouth. For example, the three-dimensional intraoral scanner 500 may be about the size of one tooth. The three-dimensional intraoral scanner 500 may be manually used to scan a patient's wisdom teeth or only a painful area of the patient's mouth without scanning the patient's entire mouth.

The three-dimensional intraoral scanner 500 may include the optical and/or ultrasonic sensors 330/335 as described above. In some instances, the three-dimensional intraoral scanner 500 may be manually moved along a patient's entire mouth to scan the patient's entire mouth. Although more time consuming than the other embodiments described herein, the smaller size of the three-dimensional intraoral scanner 500 may be useful for children and infants, for example.

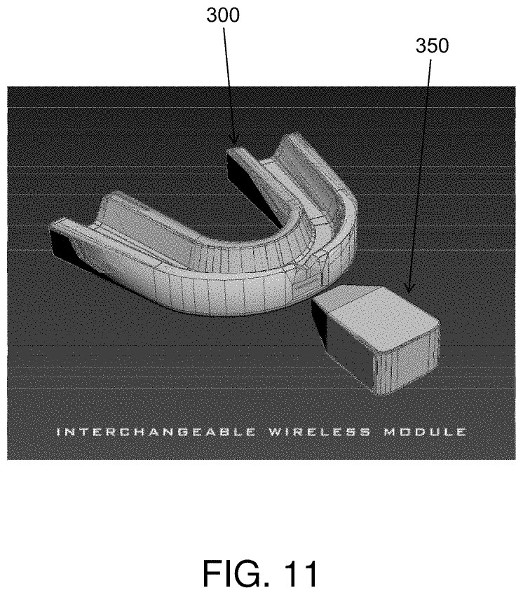

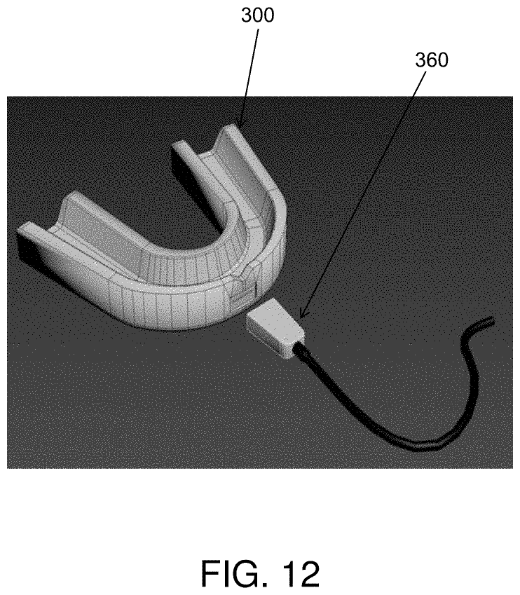

FIGS. 11 and 12 show interchangeability between a wireless module 350 and a wired connection 360. Here, the three-dimensional intraoral scanner 300 is shown as an example, but interoperability between the wireless module 350 and the wired connection 360 may be applied to any of the embodiments of the three-dimensional intraoral scanners described herein. For example, the three-dimensional intraoral scanner 300 may include a connection port, and the wireless module 350 and the wired connection 360 may both be able to connect to the connection port. In this way, the three-dimensional intraoral scanner 300 may be used with either the wireless module 350 for a wireless connection to a computer vision system or the wired connection 360 for a wired connection to the computer vision system, giving the user more flexibility.

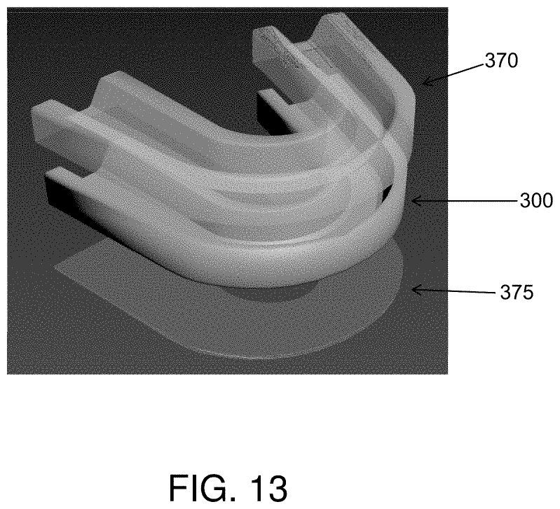

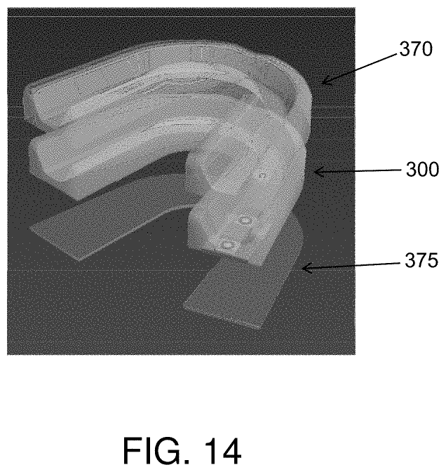

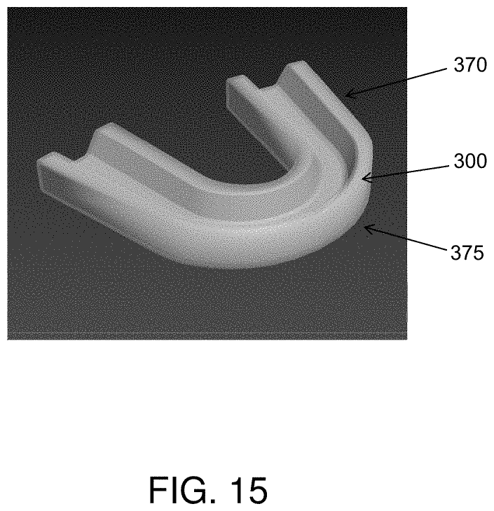

FIGS. 13-15 show covers 370/375 for the three-dimensional intraoral scanners. To protect the three-dimensional intraoral scanner 300 and for patient safety and sanitation, the covers 370/375 may be provided on the three-dimensional intraoral scanner 300. For example, an upper cover 370 may be molded to have the same shape as the trough 310 and may be press-fit onto the three-dimensional intraoral scanner 300, and a lower cover 375 may be flat to match the flat bottom of the three-dimensional intraoral scanner 300. In some embodiments, the upper cover 370 and the lower cover 375 may snap-fit together around (e.g., entirely around) the three-dimensional intraoral scanner 300 to provide a clean exterior surface for the patient. After use, the covers 370/375 may be cleaned and sterilized in an autoclave.

The covers 370/375 may be sized and shaped to fit any of the three-dimensional intraoral scanner described herein. In some embodiments, the upper cover 370 may have an opening therein to allow the wireless module 350 or the wired connection 360 to pass therethrough.

FIGS. 16 and 17 show a three-dimensional intraoral scanner 400 according to another embodiment of the present disclosure. The three-dimensional intraoral scanner 400 is similar to the three-dimensional intraoral scanner 300 shown in, for example, FIGS. 6-8 but includes both a first (or upper) trough 410 and a second (or lower) trough 420. The three-dimensional intraoral scanner 400 may include, for example, two or four sleds 320, with either one or two sleds 320 being arranged under and facing the first trough 410 and either one or two sleds 320 being arranged over and facing the second trough 420.

In some embodiments, the sled 320 may be modified to have a first group of sensors 330/335 that face into the first trough 410 and a second group of sensors 330/335 that face into the second trough 420. In such an embodiment, only one or two sleds 320 may be included in the three-dimensional intraoral scanner 400 because one sled 320 can image the patient's upper and lower teeth and oral cavity.

FIG. 18 shows examples of a point cloud of a patient's mouth, a dense point cloud of the patient's mouth, a three-dimensional representation of the patient's mouth, and a three-dimensional representation of the patient's mouth with texturing and shading.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section, without departing from the spirit and scope of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that such spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art. As used herein, the term “major component” means a component constituting at least half, by weight, of a composition, and the term “major portion”, when applied to a plurality of items, means at least half of the items.

As used herein, the singular forms “a” and “an” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the inventive concept refers to “one or more embodiments of the present disclosure”. Also, the terms “exemplary” and “example” are intended to refer to an example or illustration. As used herein, the terms “use,” “using,” and “used” may be considered synonymous with the terms “utilize,” “utilizing,” and “utilized,” respectively.

It will be understood that when an element or layer is referred to as being “on”, “connected to”, “coupled to”, or “adjacent to” another element or layer, it may be directly on, connected to, coupled to, or adjacent to the other element or layer, or one or more intervening elements or layers may be present. In contrast, when an element or layer is referred to as being “directly on”, “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein.

Although example embodiments of a three-dimensional intraoral scanner have been described and illustrated herein, many modifications and variations within those embodiments will be apparent to those skilled in the art. Accordingly, it is to be understood that a three-dimensional intraoral scanner according to the present disclosure may be embodied in forms other than as described herein without departing from the spirit and scope of the present disclosure.

What is claimed is:

1. A three-dimensional intraoral scanner comprising:
an outer body forming a trough, an outer surface of the outer body comprising a plastic material and an inner surface of the outer body at the trough comprising a transparent or translucent material, the trough being configured to fit around a row of a patient's teeth; and
a sled within the outer body and configured to move along the trough, the sled comprising a plurality of ultrasonic sensors and a plurality of optical sensors configured to image the patient's mouth.

2. The three-dimensional intraoral scanner of claim 1, wherein the sled is configured to move along the trough by using magnetic attraction and repulsion.

3. A system comprising:
the three-dimensional intraoral scanner of claim 1; and
a computer system configured to receive optical and ultrasonic images from the three-dimensional intraoral scanner and to create a three-dimensional representation of the patient's mouth.

4. A three-dimensional intraoral scanner comprising:
an outer U-shaped portion;
an inner portion connected to the outer U-shaped portion; and
a plurality of optical sensors and a plurality of ultrasonic sensors arranged on both the outer U-shaped portion and the inner portion.

5. A three-dimensional intraoral scanner comprising:
a body sized about a size of a tooth; and
a plurality of optical sensors and a plurality of ultrasonic sensors arranged in the body.
